(19) United States
(12) Patent Application Publication
(10) Pub. No.: US 2026/0144676 A1
(43) Pub. Date: May 28, 2026

(54) IDENTIFICATION OF ASPIRATED MATERIAL DURING SURGICAL PROCEDURES

(71) Applicant: Alcon Inc., Fribourg (CH)

(72) Inventors: Raphael GORDON, Ladera Ranch, CA (US); Satish YALAMANCHILI, Lake Forest, CA (US); Brian William MCDONELL, Irvine, CA (US)

(21) Appl. No.: 19/394,253

(22) Filed: Nov. 19, 2025

Related U.S. Application Data
(60) Provisional application No. 63/725,950, filed on Nov. 27, 2024.

(51) Int. Cl. A61F 9/007; A61M 1/00

(52) U.S. Cl. CPC A61F 9/00745; A61M 1/732; A61M 1/734; A61M 2205/502; A61M 2210/0612

(57) ABSTRACT

In certain embodiments, a method of identifying a composition of an aspirated material includes: controlling a surgical instrument to aspirate material through a probe that is fluidly connected with a vacuum source in an aspiration path. The method further includes estimating, during aspiration of the material, a flow rate of the material at the probe. The method further includes determining, using one or more sensors disposed along one or both of the aspiration path and infusion path, a change in pressure across the probe during aspiration. The method further includes determining a fluidic resistance of the material using the flow rate and the change in pressure, and providing feedback based, at least in part, on the fluidic resistance of the material and a reference value.

### CROSS-REFERENCE TO RELATED APPLICATIONS

This application claims the benefit of U.S. Provisional Patent Application No. 63/725,950, filed Nov. 27, 2024, which is incorporated by reference herein in its entirety, and is hereby expressly made a part of this specification.

### INTRODUCTION

Anatomically, the human eye is divided into two distinct regions: the anterior segment and the posterior segment. The anterior segment includes the lens and extends from the outermost layer of the cornea to the posterior of the lens capsule. The posterior segment of the eye includes the anterior hyaloid membrane and all of the ocular structures behind it, such as the vitreous humor, retina, choroid, and the optic nerve.

Ophthalmic surgical procedures are often classified as anterior segment surgical procedures, posterior segment procedures, or combined anterior segment and posterior segment procedures (i.e., “combined procedures”). Anterior segment surgical procedures typically include surgeries performed on the iris and/or lens, such as cataract surgery. Posterior segment surgical procedures typically include retinal and vitreoretinal surgeries. In certain cases, a patient may have pathologies of the eye requiring both anterior and posterior procedures; in such cases, a combined procedure may be performed.

Surgical procedures frequently require precision cutting and/or removing various body tissues. For example, certain ophthalmic surgical procedures require cutting and removing portions of the vitreous humor (or “vitreous”), a transparent jelly-like material that fills the posterior segment of the eye. The vitreous is composed of numerous microscopic fibrils that are often attached to the retina. Thus, any cutting and removing of the vitreous must be done with great care to avoid traction on the retina, the separation of the retina from the choroid, a tear of the retina, or cutting and removal of the retina itself.

During a vitrectomy procedure, another material may be infused into the eye (e.g., a balanced salt solution or “BSS”) to replace the aspirated vitreous and maintain the appropriate intraocular pressure within the eye. Because the other material is typically also transparent, it can be challenging for a surgeon to visually distinguish the vitreous from the other material, which can cause the procedure to be more intrusive (e.g., a longer procedure time, more material circulated through the eye, and so forth).

### BRIEF SUMMARY

The present disclosure relates generally to techniques for identifying a composition of an aspirated material during a surgical procedure. In some embodiments, a method includes controlling a surgical instrument to aspirate material through a probe that is fluidly connected with a vacuum source in an aspiration path. The method further includes estimating, during aspiration of the material, a flow rate of the material at the probe. The method further includes determining, using one or more sensors disposed along one or both of the aspiration path and an infusion path, a change in pressure (i.e., pressure delta or pressure difference) across the probe during aspiration. The method further includes determining a fluidic resistance of the material using the flow rate and the change in pressure, and providing feedback based, at least in part, on the fluidic resistance of the material and a reference value.

In some embodiments, the method further includes determining a fluidic viscosity of the material based on the fluidic resistance and a change in the flow rate, and the feedback is based on a comparison of the fluidic viscosity of the material with the reference value. In some embodiments, the reference value comprises a viscosity of a BSS provided by an infusion line. In some embodiments, the aspiration path comprises tubing, and estimating the flow rate of the material is based on a flow rate component that is attributable to compliance of the tubing.

Using the feedback, a surgeon may more efficiently manipulate the surgical instrument to selectively aspirate and cut the target material (such as vitreous), resulting in more efficient and/or less intrusive surgical procedures.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1 illustrates an exemplary surgical console of a surgical system, according to one or more embodiments.

FIG. 2 illustrates a partially cross-sectional view of an eye undergoing a procedure involving a surgical instrument and an infusion line, according to one or more embodiments.

FIG. 3 illustrates an exemplary surgical system, according to one or more embodiments.

FIG. 4 illustrates an exemplary method of identifying an aspirated material during a surgical procedure, according to one or more embodiments.

FIG. 5 illustrates an exemplary sequence of media detection during a surgical procedure, according to one or more embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

It will be readily understood that the components of the embodiments as generally described herein and illustrated in the appended Figures can be arranged and designed in a wide variety of different configurations. Thus, the following more detailed description of various embodiments, as represented in the Figures, is not intended to limit the scope of the present disclosure but is merely representative of various embodiments. While the various aspects of the embodiments are presented in the Figures, the Figures are not necessarily drawn to scale unless specifically indicated.

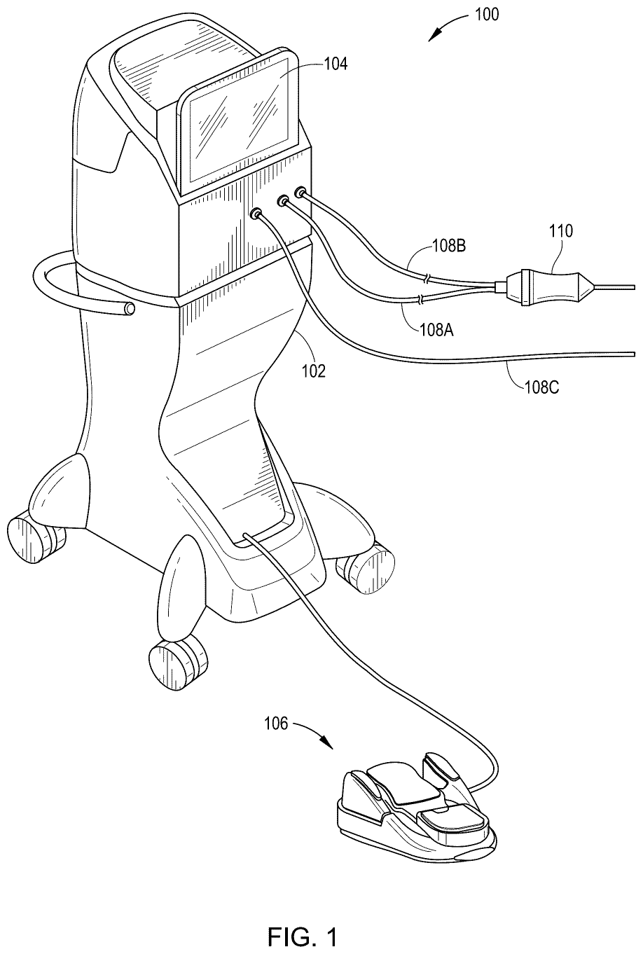

FIG. 1 illustrates an exemplary surgical console 102 (or “console 102”) of a surgical system 100, according to one or more embodiments. The surgical system 100 includes the surgical console 102 operatively connected with a surgical instrument 110, such as a vitrectomy probe handpiece, an extrusion handpiece, an ultrasonic handpiece, or the like. In some embodiments, the console 102 may be mobile, and may include wheels or casters that facilitate movement of the console 102 throughout an operating room. In other embodiments, the console 102 may be statically positioned. The console 102 may be referred to as a “base housing” and may include a plurality of subsystems whose coordinated operation enables a surgeon to perform a variety of surgical procedures, such as ophthalmic surgical procedures.

The surgical instrument 110 attaches to the surgical console 102 by one or more connection conduits 108A, 108B of the surgical system 100. Although two connection conduits 108A, 108B are shown, different numbers are also contemplated (e.g., one, three, four or more). The connection conduits 108A, 108B may be operated to supply high and low fluid pressures, electrical power, optical power, control signals, and so forth to the surgical instrument 110. In some implementations, one or both of the connection conduits 108A, 108B are formed of lengths of tubing forming fluid conduits that convey fluids, such as air, saline, removed material (e.g., vitreous or lens material), substance, or others, between the surgical instrument 110 and one or more subsystems of the console 102. In some embodiments, the connection conduits 108A and/or 108B may each include a plurality of lumens that enable multiple fluids to be conveyed between the surgical instrument 110 and the console 102. In some embodiments, one or both of the connection conduits 108A, 108B may be formed as conductive cables, supplying electrical power to the surgical instrument 110 and/or conveying control signals between the surgical instrument 110 and the console 102. For example, the surgical instrument 110 may include a switch or other control mechanism (e.g., a button, slider, or roller) operable by a surgeon to control a cutting action of the surgical instrument 110, to enable aspiration of cut material, to enable infusion of a substance, and so forth. Thus, in some embodiments, the surgical instrument 110 forms part of a vitrectomy subsystem described herein.

The surgical console 102 may be further connected with one or more additional connection conduits that provide functionality during a surgical procedure. As shown, the surgical console 102 is connected with a connection conduit 108C that is operated to supply an infusion material (e.g., a fluid or a gel) during the surgical procedure, which will be discussed in greater detail below. In some implementations, the connection conduit 108C is formed of a length of tubing forming a fluid conduit.

The console 102 may include an associated display 104 that is configured to visually display information relating to operation and performance of the surgical system 100 during a surgical procedure (e.g., a vitrectomy procedure). Although not shown, the console 102 may include other feedback devices (e.g., audio speakers, haptic motors) that provide information during the surgical procedure.

In some embodiments, the surgical system 100 further includes an input device 106 (also referred to as an “electronic accessory”) that may be any device that is capable of receiving commands from the user of the console 102 to operate the surgical instrument 110 and/or to provide control to other components of the surgical console 102. In FIG. 1, the input device 106 is illustrated as a foot controller, however, other types of input devices are also within the scope of the disclosure, such as a touchscreen integrated with the display 104, physical buttons included on the surgical console 102, and so forth. In one example, the user provides a command to the input device 106 (e.g., a surgeon using his or her foot to manipulate a pedal of the foot controller), which is transmitted to a controller of the console 102 (e.g., by an input subsystem of the console 102). In response, the controller transmits instructions to enable and/or control the operation of the surgical instrument 110 (e.g., by a handpiece subsystem and/or aspiration subsystem of the console 102) based on the user command.

In some embodiments, the input device 106 includes one or more feedback devices, such as a visual display, audio speakers, and/or haptic motors that provide information during a surgical procedure. In some embodiments, and as will be discussed in greater detail below, the display 104 and/or other feedback devices included in the console 102 and/or the input device 106 may provide feedback that indicates a composition of a material that is being aspirated through a probe of the surgical instrument 110 (e.g., through a needle of the vitrectomy probe, a soft-tip cannula, an extrusion needle, and so forth). Providing this feedback in real-time enables a surgeon to more quickly identify the material that is encountered by the probe, to identify target material (e.g., vitreous or lens material) for aspiration, and to selectively and completely aspirate the target material. In this way, the surgical procedure may be more accurate while being minimally invasive, requiring less time to complete, less fluid to be circulated through the eye, and/or less recovery time for the patient.

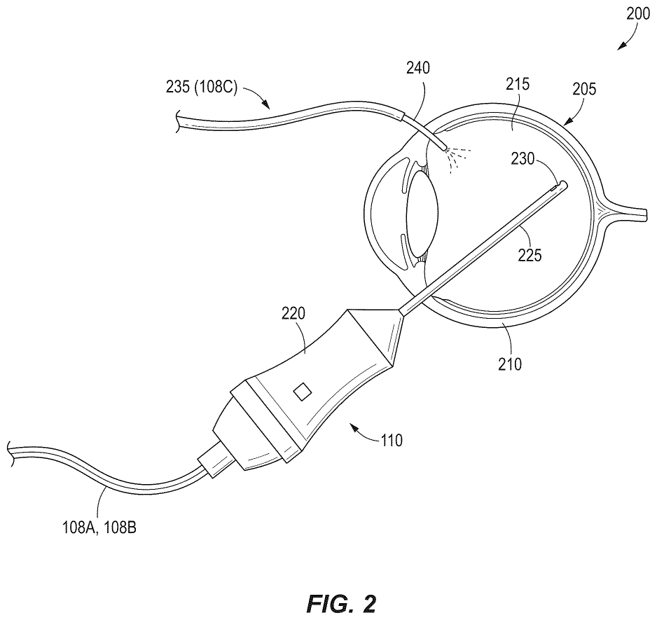

FIG. 2 illustrates a partially cross-sectional view 200 of an eye 205 undergoing a procedure involving the surgical instrument 110 and an infusion line 235, according to one or more embodiments. The features illustrated in the view 200 may be used in conjunction with other embodiments, e.g., using various components of the surgical system 100 of FIG. 1.

The surgical instrument 110 comprises a body 220 and a probe 225 extending from a distal end of the body 220. Note that, as described herein, a distal end, segment, or portion of a component refers to the end, segment, or portion that is closer to a patient's body during use of the component. On the other hand, a proximal end, segment, or portion of the component refers to the end, segment, or portion that is distanced further away from the patient's body and is in proximity to, for example, the surgical console 102.

Both the probe 225 and the infusion line 235 may be operatively connected to a console, e.g., to the console 102 of FIG. 1 via the connection conduits 108A, 108B, 108C that are connected to a proximal end of the body 220. During a vitrectomy procedure, the probe 225 and the infusion line 235 are inserted through incisions formed in the sclera 210 (e.g., sclerotomies) and into a vitreous chamber 215 of the eye 205. Although not shown, a fiber optic light source (e.g., an endoilluminator) may also be inserted through a similar incision and used to illuminate the interior of the eye 205 during the procedure.

The probe 225 may have any suitable implementation for cutting and aspirating vitreous and/or other materials. The probe 225 defines a port 230 near its distal end, through which vitreous is aspirated into the probe 225. In some embodiments, the probe 225 comprises a reciprocating cutting assembly, e.g., having a hollow outer cutting member and a hollow inner cutting member arranged coaxially with and movably disposed within the hollow outer cutting member. The port 230 may extend radially through the outer cutting member. During operation of the reciprocating cutting assembly, vitreous and/or membranes are aspirated into the port 230, and the inner member is actuated to close the port 230. Closing the port 230 causes complementary cutting surfaces of the inner cutting member and the outer cutting member to cut the vitreous and/or membranes, which is then aspirated through the inner cutting member.

In other embodiments, the probe 225 comprises a laser cutting assembly, where laser pulses are generated (typically, proximal to the probe 225 such as at the surgical console 102) and propagated through the probe 225 to cause disruption of the vitreous within the path of the laser pulses. Disruption refers to the breaking down of the tissue by rapid ionization of molecules thereof. During operation of the laser cutting assembly, vitreous and/or membranes are aspirated into the port 230, and the laser pulses disrupt the aspirated material. Other types of the cutting assembly are also contemplated, such as an ultrasonic cutting assembly that delivers ultrasonic energy to liquefy vitreous.

The infusion line 235 is attached to an infusion cannula 240 that is used to deliver replacement fluid or irrigation fluid into the vitreous chamber 215 during vitrectomy procedures. In some embodiments, a saline solution (e.g., a balanced salt solution or BSS) is infused into the eye 205 via the infusion line 235 to replace the aspirated vitreous and maintain the appropriate intraocular pressure within the eye 205. Other types of infusion materials are also contemplated, which in some cases may be introduced into the eye 205 through means other than the infusion line 235 (e.g., using a needle and syringe). These other infusion materials may be liquids or gel-like, such as Brilliant Blue G ophthalmic solution, indocyanine green (“ICG”), viscoelastic substances such as hyaluronic acid, and so forth. In some embodiments, a pressure level of the irrigation fluid may be controlled dynamically during operation by the surgical system 100, e.g., using input signals provided by a foot controller.

In some embodiments, the material infused into the eye 205 may be a transparent fluid, such that it may be challenging for a surgeon to visually distinguish the vitreous from the infused material during the procedure. As a result, the procedure may be unnecessarily intrusive, requiring a longer procedure time, more material to be circulated through the eye, and so forth. In various embodiments described herein, the surgical system 100 identifies the aspirated material by determining a fluidic resistance of the material (e.g., a viscosity) using a flow rate at the probe and a change in pressure across the probe. In some embodiments, the surgical system 100 adjusts or refines an initial value of the flow rate by estimating, using a predefined model, a flow rate component that is attributable to the compliance of tubing and/or other components connecting the surgical console 102 and the surgical instrument 110. The surgical system 100 provides real-time feedback to the surgeon based, at least in part, on a comparison of the fluidic resistance of the material with a reference value. Using the feedback, the surgeon may more efficiently manipulate the surgical instrument 110 and selectively aspirate and cut the target material (such as vitreous), resulting in more efficient and/or less intrusive procedures. Further, where non-cutting instruments are used, the detection of vitreous can be used to mitigate or prevent a condition where unseen residual vitreous is pulled into the instrument resulting in traction, especially if the instrument is moved.

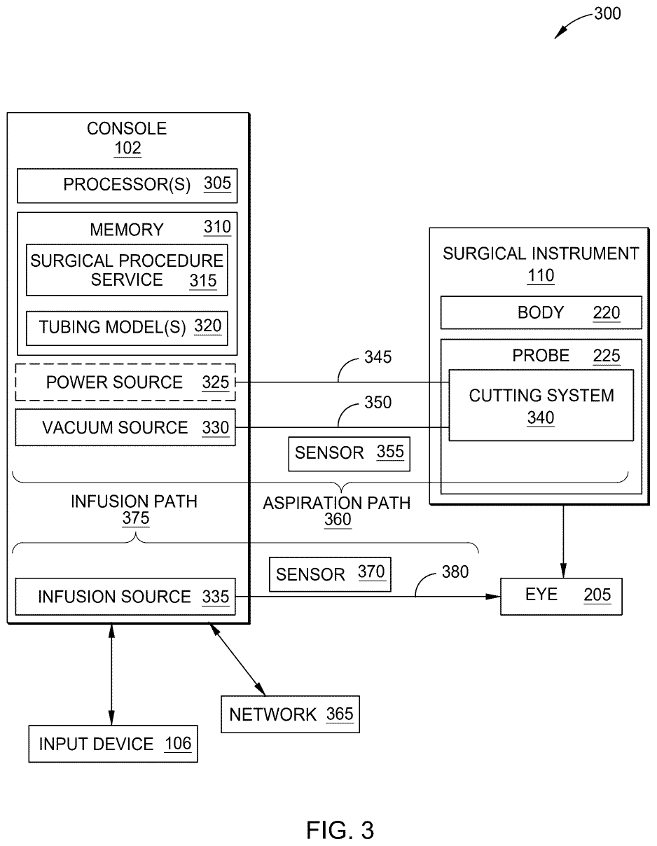

FIG. 3 illustrates an exemplary surgical system 300, according to one or more embodiments. The features illustrated in FIG. 3 may be used in conjunction with other embodiments. For example, the surgical system 300 may represent one example implementation of the surgical system 100 of FIG. 1.

The surgical console 102 comprises one or more processors 305 and a memory 310. The one or more processors 305 may include any type(s) of general-purpose processor and/or processor specifically designed for performing various functions described herein, such as an application-specific integrated circuit (“ASIC”). The one or more processors 305 may be, or may include, a microprocessor, a microcontroller, an embedded microcontroller, a programmable digital signal processor, or any other programmable device operable to execute instructions stored in the memory 310 for operating the surgical console 102, or combinations thereof. In some instances, the one or more processors 305 may also be, or may include, a programmable gate array, programmable array logic, or any other device of combinations of devices operable to process electronic signals.

The memory 310 can be any type of storage device or non-transitory computer-readable medium, such as random-access memory (“RAM”) or read-only memory (“ROM”), which is operable to receive, store, or recall data, including, but not limited to, electronic, magnetic, or optical memory, whether volatile or non-volatile. The memory 310 stores instructions executed by the one or more processors 305. In example embodiments, functionality disclosed herein can be provided by the one or more processors 305 and the memory 310 (i.e., software-based), by other circuitry of the one or more processors 305 (i.e., hardware-based), or by a combination thereof. The memory 310 may include code stored thereon. The code may include instructions that may be executable by the one or more processors 305. The code may be created, for example, using any programming language, including but not limited to, C, C++, Java, Python, Rust, or any other programming language (including assembly languages, hardware description languages, and database programming languages). In some embodiments, the code may be a program that, when executed by the one or more processors 305, controls cutting and aspiration functions of the surgical instrument 110, estimates and/or receives parameters related to the aspirated material, accesses a predefined model, identifies the aspirated material, provides real-time feedback to the surgeon, and so forth. As shown, the memory 310 includes a surgical procedure service 315 that represents a single program providing the described functionality. In other embodiments, the code may be provided as multiple programs that cooperate to perform the various functions of the surgical console 102. As used herein, references to functionality provided by the surgical procedure service 315 are intended to encompass embodiments having a single program, multiple programs, combinations of program(s) and hardware-provided functionality, and so forth.

In some embodiments, the surgical console 102 further comprises a power source 325, a vacuum source 330, and an infusion source 335. The power source 325 provides, via one or more power connections 345, operative power to a cutting system 340 of the surgical instrument 110. In some embodiments, the cutting system 340 comprises a reciprocating cutting assembly and the power source 325 may provide pneumatic power and/or electrical power to operate the cutting system 340. In other embodiments, the cutting system 340 comprises a laser cutting assembly and the power source 325 may provide optical power and/or electrical power to operate the cutting system 340. In yet other embodiments, the cutting system 340 comprises an ultrasonic cutting assembly and the power source 325 may provide electrical power to operate the cutting system 340.

In some embodiments, the cutting system 340 is arranged fully or partly in the probe 225. The cutting system 340 may further be arranged in the body 220 of the surgical instrument 110, and/or external to the surgical instrument 110. For example, the reciprocating cutting assembly may include actuator components in the body 220 that are operatively connected with an outer cutting member and/or an inner cutting member that extend into the probe 225. In another example, the laser cutting assembly may include a laser source external to the body 220 (e.g., within the power source 325 or along the power connections 345), and an optical fiber that propagates and/or amplifies laser pulses from the laser source and extends into the probe 225.

The vacuum source 330 supplies vacuum via one or more vacuum connections 350 to the surgical instrument 110 (e.g., to the cutting system 340) to aspirate material into and through the probe 225. The vacuum source 330 may have any suitable implementation, such as one or more pumps. In this way, the surgical system 300 defines an aspiration path 360 to the eye 205 that includes the vacuum source 330, the one or more vacuum connections 350, and the probe 225. The aspiration path 360 may include one or more additional components. As will be discussed in greater detail below, in some embodiments a sensor 355 is disposed along the aspiration path 360 that indicates a change in pressure occurring across the probe 225 during aspiration. The vacuum source 330 may further be connected to a vacuum collector that receives the aspirated material.

The power connections 345 and the vacuum connections 350 represent examples of the connective conduits 108A, 108B of FIG. 1. In some embodiments, the power connections 345 comprise one or more of: tubing (e.g., rigid tubing and/or flexible tubing), electrical conductors (e.g., wires or cabling), and optical conductors (e.g., optical fibers).

The infusion source 335 provides, via the infusion line 235, replacement fluid or irrigation fluid to the eye 205 during the surgical procedure. The infusion source 335 may have any suitable implementation, such as one or more pumps. In this way, the surgical system 300 defines an infusion path 375 to the eye 205 that includes the infusion source 335 and one or more infusion connections 380, which in some cases includes the infusion line 235 and the infusion cannula 240. In some embodiments, a sensor 370 is disposed along the infusion path 375 that indicates a change in pressure during infusion.

In some embodiments, the surgical procedure service 315 transmits control signals to one or more of the power source 325, the vacuum source 330, the infusion source 335, and/or the cutting system 340 to control one or more operational parameters of the corresponding components during the procedure. For example, the control signals may control whether operation of the particular component is enabled or disabled, a cutting rate, pump speeds, characteristics of laser pulses, and so forth. In some embodiments, the control signals may be dynamically controlled based on inputs provided by the surgeon, e.g., using the input device 106.

During the procedure, the surgical procedure service 315 estimates and/or receives parameters related to the aspirated material and/or to the infused material, and identifies the aspirated material based on these parameters. In some embodiments, the parameters include pressures and flow rates corresponding to various points along the aspiration path. Using a change in pressure and a flow rate, a fluidic resistance (or viscosity) of the aspirated material can be determined, and compared with a reference value corresponding to a known material such as BSS. In one non-limiting example implementation, along the infusion path 375 the surgical procedure service 315 measures a pressure and flow rate at the console 102 (e.g., at or near the infusion source 335), estimates a pressure at a proximal end of the infusion cannula 240, and estimates a flow rate through the infusion cannula 240. The surgical procedure service 315 estimates a pressure at the eye 205. Along the aspiration path 360 the surgical procedure service 315 estimates a flow rate through the surgical instrument 110, estimates a pressure at a proximal end of the surgical instrument 110, and measures a pressure and flow rate at the console 102 (e.g., at or near the vacuum source 330).

In some embodiments, each of the vacuum source 330 and the infusion source 335 includes a respective pump having instrumentation that provides flow rate measurements to the surgical procedure service 315. Each of the vacuum source 330 and the infusion source 335 may further include a pressure transducer that provides pressure measurements to the surgical procedure service 315.

During aspiration, most of the pressure drop occurs at the probe 225. In some embodiments, the fluidic resistance may be determined using pressure measurements obtained at the surgical console 102. However, it may be beneficial to position sensors in more distal locations than the pressure transducers at the surgical console 102 to provide improved accuracy when measuring relevant pressures. In some embodiments, a sensor 355 is disposed along the aspiration path 360 at a more distal location than the surgical console 102, for example, disposed at the vacuum connections 350 and/or at the surgical instrument 110. In some embodiments, the sensor 355 is disposed in line with the tubing, near a connection of the tubing with the surgical instrument 110, at a proximal end of the body 220, at a distal end of the body 220, or within the eye to measure an intraocular pressure. For example, the sensor 355 may be disposed at the proximal end of the body 220 where the cutting system 340 comprises a reciprocating cutting assembly at the distal end of the body 220. In another example, the sensor 355 may be disposed at the distal end of the body 220 where the cutting system 340 comprises a laser cutting assembly not including moving parts in the distal end of the body 220.

In some embodiments, the sensor 355 comprises a pressure sensor of any suitable type, e.g., a pressure transducer. In other embodiments, the sensor 355 comprises another type of sensor providing measurements usable for a fluidic resistance determination, such as a flow sensor having any suitable sensing technology (e.g., ultrasonic flow meter, magnetic flow meter, and so forth).

The surgical procedure service 315 determines a fluidic resistance of the aspirated material using the flow rate and the pressure, which may be determined at various locations according to embodiments described herein. In some embodiments, the fluidic resistance of the aspirated material is determined as:

$$R = \frac{Δ P}{Q} \qquad ( 1 )$$

where R represents the fluidic resistance, ΔP represents a difference between a pressure measurement of the sensor 355 and a reference pressure (e.g., a measurement of intraocular pressure), and Q represents a flow rate measurement. In some embodiments, one or more of the terms of Equation (1) may be adjusted. For example, a pressure measurement obtained by the sensor 355 at one position along the aspiration path 360 may be shifted, scaled, etc. to estimate a pressure at another position along the aspiration path 360.

In some embodiments, the aspirated material may include a Newtonian fluid or a non-Newtonian fluid. The fluidic resistance may be constant or a linear or non-linear function of pressure and flow rate. In some embodiments, a sensor 370 is arranged along the infusion path 375 to obtain pressure measurements that may be used in conjunction with a tubing model 320 for the infusion path, an infusion flow rate, and a resistance of the infusion cannula 240 to estimate an intraocular pressure of the eye 205.

The surgical procedure service 315 compares the determined fluidic resistance (or a value derived therefrom) with a reference value to identify a composition of the aspirated material. In some embodiments, the reference value corresponds to the fluidic resistance or a fluidic viscosity of a particular material, which in some cases is the same material as the infusion material supplied using the infusion line 235. For example, in some embodiments, the reference value comprises a fluidic viscosity of BSS. In other embodiments, the reference value is an interpolated value corresponding to a composition of multiple materials (e.g., 90% BSS and 10% vitreous), an adjusted value (e.g., the fluidic resistance of BSS+10%), and so forth.

Based on the comparison, the surgical procedure service 315 provides feedback indicating the composition of the aspirated material. The feedback may be provided using any suitable devices, such as a visual display, audio speakers, and/or haptic motors that provide real-time information to the surgeon during the procedure. The feedback may be provided using the console 102 (e.g., the display 104), the input device 106, the surgical instrument 110, and so forth.

Continuing the example of detecting vitreous from BSS, vitreous is more viscous than BSS and generally causes the determined fluidic resistance of the aspirated material to increase. Other target and reference materials that may be distinguished by different fluidic resistances are also contemplated.

In some embodiments, the feedback indicates the mere presence of vitreous in the aspirated material. For example, when the determined fluidic resistance exceeds the reference value, the surgical procedure service 315 may generate an audio tone. In some embodiments, the feedback indicates a proportion of vitreous in the aspirated material. For example, as the determined fluidic resistance increases beyond the reference value, indicating a greater proportion of vitreous in the aspirated material, the surgical procedure service 315 may generate an audio output having proportionally increasing characteristic(s), such as volume, frequency, rate, and so forth. In some embodiments, the feedback indicates an amount of vitreous in the aspirated material. For example, the surgical procedure service 315 may access a value indicating a target volume of vitreous, and calculate a volume of aspirated vitreous based on the determined values of fluidic resistance. The surgical procedure service 315 may generate an audio output having proportionally increasing characteristic(s) as the calculated volume increases toward the target volume.

While audio outputs have been described, other feedback arrangements are also contemplated, such as decreasing characteristics, different types of feedback, providing multiple types of feedback (e.g., combination of audio and visual feedback), and so forth. In one non-limiting example, the display 104 may visually display a sequence of lights: a first light having a first color (e.g., blue) that is illuminated when BSS is encountered, and a second light having a second color (e.g., red) that is illuminated when vitreous is encountered. In another non-limiting example, a color of a border displayed on the display 104 may be changed depending on whether vitreous is encountered (e.g., blue or red). In another non-limiting example, a light color of an endoilluminator may be changed depending on whether vitreous is encountered (e.g., blue or red).

In a first example implementation, the surgical procedure service 315 receives a measurement of a flow rate of the aspirated material at the vacuum source 330, and estimates the flow rate of the aspirated material at the probe 225 to be the same. The surgical procedure service 315 determines a fluidic resistance of the aspirated material using at least the flow rate, a pressure measurement acquired by the sensor 355, and a pressure measurement received from the vacuum source 330. In one example, the flow and pressure measurements are obtained at the proximal end of the vacuum connection(s) 350 and/or infusion connection(s) 380 at the surgical console 102. In such embodiments, one or more pumps of the surgical system 300 are the source(s) of pressure, and the flow measurements may be estimated from a model of the pump efficiency, which in some cases is based on pump pressures. The pressure measurements are obtained in the path between the pump and the vacuum connection(s) 350 and/or infusion connection(s) 380. Based on the model(s), the estimated flow and pressures at the surgical instrument 110 can be determined.

The performance of the infusion-side pump and the aspiration-side pump can be measured and modeled, e.g., Q=V η(P, ω) ω, where Q represents a flow rate (volume per time), V represents a nominal pump volume (volume per radian), n represents efficiency as a function of pressure (P) and angular frequency (ω, radians per time). The fluidic resistance of the infusion cannula 240 and the surgical instrument 110 can be measured, possibly as a function of pressure and flowrate. The fluidic resistance of the surgical instrument 110 may also vary with other operating parameters. For example, the vitrectomy probe performance would vary depending on whether the cutter is on or off, a cut rate and so forth, e.g., R_{probe}=R(P, Q, f_{cutter}), where R is a function of the pressure (P), the flow rate (Q) and a frequency of the cutter (f_{cutter}). The compliance of the eye 205 can be measured, possibly as a function of pressure, e.g., C(P). The viscosity of BSS can be measured.

Using some or all of these parameters, the surgical procedure service 315 may estimate intraocular pressure, flow through the infusion cannula 240, pressure at the proximal end of the surgical instrument 110, resistance of the surgical instrument 110, flow through the surgical instrument 110, or an expected flow through the surgical instrument 110 for a reference material (e.g., BSS). For example, the surgical procedure service 315 may estimate the eye pressure using the infusion-side model(s), and the proximal instrument pressure and flow are estimated using the aspiration-side model(s). In some embodiments, the material that is aspirated by the surgical instrument 110 may be detected based on comparing the estimated resistance to the baseline fluidic resistance of BSS. In other embodiments, and using the model(s), the material may be detected based on an estimated flow compared to the baseline flow estimate (assuming the fluidic resistance of BSS).

In some embodiments, the fluidic model parameters are determined based on measurements obtained during priming of the infusion system through measurement during priming.

Notably, use of the model(s) along the infusion path 375 and the model(s) along the aspiration path 360 provide redundancy in the obtained measurements. In some embodiments, the measurements obtained along one path (e.g., the aspiration path 360) may be used by default, and responsive to one or more criteria, the measurement obtained along the other path (e.g., the infusion path 375) may be used. Although flow estimates along the infusion path 375 generally benefit from constant, gas-free fluid and minimal pressure variation, such estimates may be affected by volume changes caused by leakage, manipulation, or deformation occurring within the eye 205. Further, although flow estimates along the aspiration path 360 generally benefit from direct attachment to the surgical instrument 110, such estimates may be affected by the presence of gas in the aspirated material and a larger range of dynamic pressure changes. In other embodiments, the measurements obtained along each path may be used and/or combined in other ways. In some cases, the measurements may be compared against each other, may be averaged together, and so forth. For example, the redundant flow and pressure estimates of the surgical instrument 110 may be calculated from the infusion model(s) and aspiration model(s) and integrated together based on one or more rules (e.g., a larger of the two flow estimates is selected to calculate the fluidic resistance) to provide an estimate of the viscosity of the aspirated material.

In the first example implementation, an assumption is made that the flow rate is the same at the vacuum source 330 and at the probe 225 (or at least, any difference between the two flow rates is negligible). However, in a second example implementation, a compliance of the tubing and/or characteristics of other components of the vacuum connections 350 is reflected in the estimation of the flow rate at the probe 225.

In some embodiments, the surgical procedure service 315 accesses one or more tubing models 320 indicating the compliance of the tubing along the aspiration path 360 and/or along the infusion path 375. In some embodiments, the tubing model(s) 320 may reflect one or more characteristics of other components of the vacuum connections 350 and/or the infusion connection 380 that may affect the flow rate into the vacuum source 330 (or out of the infusion source 335). The compliance of the tubing refers to a deformation or a decrease in an internal volume of the tubing based on the pressure. For example, the vacuum source 330 operates at a first vacuum pressure and, responsive to the probe 225 encountering a more viscous material (e.g., vitreous), the pressure decreases in the tubing and the tubing is deformed. This deformation causes an additional compliance flow to be supplied to the vacuum source 330 from the tubing. To maintain the flow rate through the tubing, the vacuum source 330 typically operates at a second vacuum pressure less than the first vacuum pressure.

The tubing model(s) 320 representing the vacuum connections 350 and/or the infusion connection 380 may be modeled as fluidic transmission lines. In some embodiments, the tubing model(s) 320 are a zero-order approximation with assumptions of no pressure drop and equal inflow and outflow (e.g., inflow and outflow rates) at distal and proximal ends of the connection(s). In some embodiments, the tubing model(s) 320 represent only a fluid compliance of the vacuum connections 350 and/or the infusion connection 380. In other embodiment, the tubing model(s) 320 may be more complex, e.g., including the fluidic properties of the vacuum connections 350 and/or the infusion connection 380 (e.g., fluid compliance, fluid impedance, and/or fluid inertance). Fluid impedance may refer to a fluid resistance, fluid capacitance, and/or fluid inductance of the vacuum connections 350 and/or the infusion connection 380. Further, the tubing model(s) 320 may be implemented as a lumped model or a distributed model (e.g., multiple fluid resistance elements, fluid compliance elements, and/or fluid inertance elements). The tubing model(s) 320 may be a linear model or a non-linear model. Further, the fluid may be assumed to be Newtonian or non-Newtonian.

Thus, the tubing model 320 correlates internal flow rate values to pressure values (or to changes in the flow rate or in the pressure). The compliance of the tubing also depends on the material and the geometry of the tubing (e.g., a gauge defining an inner diameter, outer diameter, and wall thickness), as well as on the aspirated material. In some embodiments, the tubing model 320 may further depend on a gauge of the tubing. In some embodiments, the tubing model 320 may further depend on which material(s) (e.g., a reference material such as BSS, a target material such as vitreous) are being aspirated. In some embodiments, the tubing model 320 may further depend on the presence of voids (e.g., gas bubbles) within the aspirated material.

Using the tubing model 320, the surgical procedure service 315 estimates a flow rate component that is attributable to the compliance of the tubing, and estimates the flow rate of the material at the probe 225 by subtracting the flow rate component from an initial value of the flow rate.

In some embodiments, the tubing model 320 is stored in the memory 310 of the console 102. In some embodiments, the surgical procedure service 315 retrieves the tubing model 320 based on identification information for one or more components included in the aspiration path 360. For example, the memory 310 may store a plurality of different tubing profiles corresponding to different gauges and/or other characteristic(s), and the tubing model 320 of a particular tubing profile is selected based on the identification information.

In another example, the surgical procedure service 315 retrieves the tubing model 320 from a network 365. The network 365 may have any suitable implementation, such as one or more wide area networks (WANs), one or more local access networks (LANs), or combinations thereof. The network 365 comprises infrastructure for communicative capability, such as conductive cabling, wireless transmission, optical transmission, and so forth. The network 365 may further comprise one or more electronic devices providing network functionality and/or services to the network 365, such as routers, firewalls, switches, gateway computers, edge servers, and so forth. In some embodiments, an electronic device is communicatively coupled with the surgical console 102 through the network 365.

The identification information may be provided in any suitable manner. In some embodiments, the surgeon or technician may provide input (e.g., using the input device 106 or the console 102) to specify a particular model of tubing, the gauge, and so forth. In other embodiments, the tubing may be provided with an electronic tag such as a Radio Frequency Identification (RFID) tag that is read by the console 102 to identify the tubing. In other embodiments, the tubing includes visual indicators that may be unencoded (e.g., a model or serial number in plaintext) or encoded (e.g., a bar code or Quick Response (QR) code). The visual indicators may be read by the console 102 to identify the tubing.

In some embodiments, the surgical procedure service 315 uses the identification information to retrieve the tubing model 320, e.g., from the memory 310 or from the network 365. In other embodiments, the tubing model 320 or its characteristics (e.g., a distinct set of coefficients) may be included within the identification information.

In a third example implementation, an assumption may be made that the surgical system 300 is a closed system and leakage is negligible, such that the inflow supplied to the eye 205 by the infusion line 235 is equal to the outflow through the vacuum connections 350. Referencing one or more measurements to components along the infusion path (e.g., the infusion source 335 and/or the infusion line 235) may be beneficial, as the infusion path tends to be better controlled than the aspiration path 360 due to the absence of air, smaller variation in pressure including the absence of vacuum, shorter time constraints, and so forth.

In some embodiments, the surgical procedure service 315 receives a measurement of the flow rate at the infusion source 335, and a measurement of pressure using the sensor 355. The surgical procedure service 315 accesses flow characteristics for a reference material (e.g., BSS), which may be referenced to pressure values. The surgical procedure service 315 estimates eye pressure using the infusion-side model(s), and estimates the flow rate at the proximal side of the probe 225 by determining an expected flow rate through the probe 225 using the flow characteristics.

The surgical procedure service 315 further receives a measurement of the flow rate at the vacuum source 330, and determines a fluidic resistance of the aspirated material based on the difference between the expected flow rate and the measured flow rate at the vacuum source 330. In some embodiments, the fluidic resistance is determined based on a ratio of the actual flow rate to the expected flow rate (e.g., assuming BSS or other reference material). In other embodiments, the fluidic resistance is determined based on a subtractive difference of the actual flow rate with the expected flow rate. Adjustments to the ratio or the subtractive difference when determining the fluidic resistance are also contemplated. Further, in some embodiments, the surgical procedure service 315 determines a fluidic viscosity of the aspirated material and compares with a reference value (e.g., a fluidic viscosity of BSS) to better differentiate aspirated material. fluids from BSS.

In the third example implementation, any compliance of the tubing and/or characteristics of other components of the vacuum connections 350 is not reflected in the expected flow rate. However, in a fourth example implementation, the surgical procedure service 315 accesses the tubing model 320 and determines a flow rate component of the measured flow rate at the vacuum source 330 that is attributable to the compliance of the tubing. Subtracting the flow rate component from the measured flow rate at the vacuum source 330 improves the accuracy of the ratio, difference, etc. that is used to determine the fluidic resistance of the aspirated material.

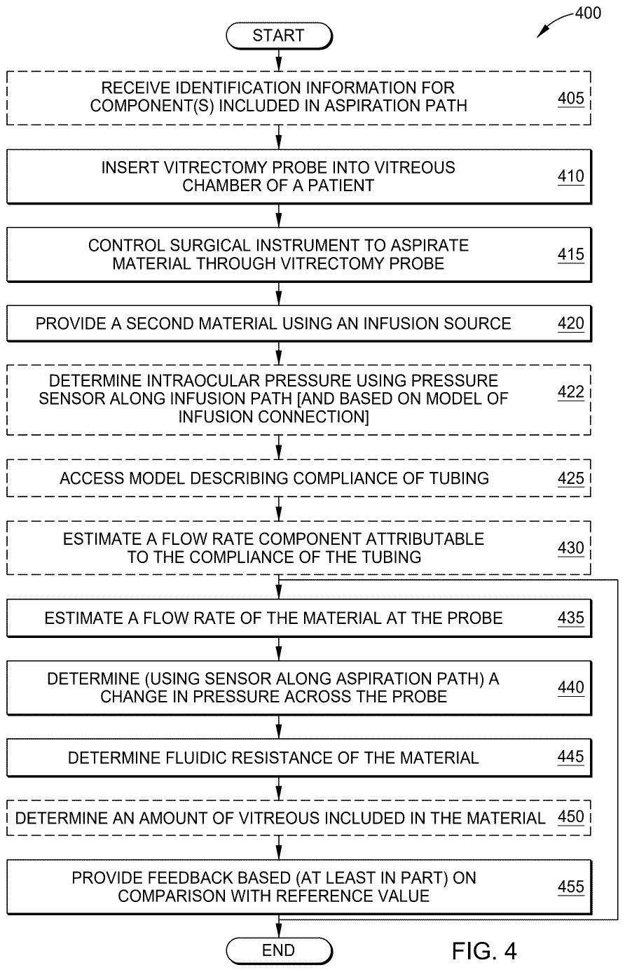

FIG. 4 illustrates an exemplary method 400 of identifying an aspirated material during a surgical procedure, according to one or more embodiments. The method 400 may be used in conjunction with other embodiments, e.g., performed using the surgical procedure service 315 of FIG. 3.

Method 400 begins at an optional block 405, where the surgical procedure service 315 receives identification information for component(s) included in an aspiration path. In some embodiments, the surgical procedure service 315 additionally or alternately receives identification information for component(s) included in an infusion path. In some embodiments, the identification information is provided by user input to the console 102. In other embodiments, the identification information is provided by an electronic tag or visual indicators that are read by the console 102.

At block 410, a surgeon inserts a vitrectomy probe into a vitreous chamber of a patient. At block 415, the surgical procedure service 315 controls the surgical instrument to aspirate material through the vitrectomy probe. In some embodiments, the surgical procedure service 315 receives input from a surgeon by an input device connected to the console 102, and controls one or more operational parameters of the surgical instrument based on the input. At block 420, the surgical procedure service 315 provides a second material (e.g., BSS) using an infusion source.

At an optional block 422, the surgical procedure service 315 calculates an intraocular pressure using a pressure sensor along the infusion path 375, and optionally based on a model of the infusion connection 380.

At an optional block 425, the surgical procedure service 315 accesses a model indicating a compliance of tubing arranged between the vitrectomy probe and a vacuum source of the console 102. In some embodiments, the model is stored in the console 102 and retrieved based on the identification information. In other embodiments, the surgical procedure service 315 retrieves the model from a network. In yet other embodiments, the model or its characteristics may be included within the identification information. At an optional block 430, the surgical procedure service 315 estimates a flow rate component that is attributable to the compliance of the tubing.

At block 435, the surgical procedure service 315 estimates a flow rate of the aspirated material at the probe. In some embodiments, the estimated flow rate is based on a measured flow rate at the vacuum source or at the infusion source. In other embodiments, the estimated flow rate is based on a measurement of a sensor disposed along an aspiration path. In some embodiments, estimating the flow rate comprises subtracting the flow rate component from an initial value of the flow rate.

At block 440, the surgical procedure service 315 determines, using a sensor disposed along the aspiration path, a change in pressure across the probe. In some embodiments, the sensor is disposed along the aspiration path at a more distal location than the console 102, for example, at the vacuum connections and/or at the surgical instrument. At block 445, the surgical procedure service 315 determines a fluidic resistance of the material using the flow rate and the change in pressure.

At optional block 450, the surgical procedure service 315 determines an amount of vitreous included in the material. In some embodiments, the surgical procedure service 315 determines the presence of vitreous in the aspirated material. In some embodiments, the surgical procedure service 315 determines a proportion of vitreous in the aspirated material. In some embodiments, the surgical procedure service 315 determines a total amount of vitreous in the aspirated material.

At block 455, the surgical procedure service 315 provides feedback based (at least in part) on a comparison of the fluidic resistance of the aspirated material with a reference value. In some embodiments, the reference value corresponds to the fluidic resistance of a particular material, which may be the same as the infusion material. For example, in some embodiments, the reference value comprises a fluidic resistance of BSS.

In some embodiments, the feedback indicates one of: the presence of vitreous in the aspirated material, the proportion of vitreous in the aspirated material, and the total amount of vitreous aspirated. In some embodiments, the feedback is provided using a visual display, audio speakers, and/or haptic motors included in the input device or the console. In some embodiments, the characteristics of the feedback are controlled based on the amount of vitreous included in the aspirated material, e.g., proportionally increasing characteristic(s) such as volume, frequency, rate, and so forth. The method 400 returns from block 455 to block 435 during the surgical procedure.

While the embodiments are described with respect to the specific example of a vitrectomy procedure, the techniques described may be used in conjunction with any surgical or clinical procedure that aspirates different materials that are capable of being distinguished from each other using their fluidic resistances.

For example, the techniques may also be used in a posterior vitreous detachment (PVD) step of a procedure, where visualization of the vitreous may be difficult. In this step, a surgeon may disable the cutting assembly and attempt to aspirate vitreous into the probe thereby causing an occlusion and achieving a purchase on the vitreous. As it is challenging for the surgeon to determine whether vitreous has been encountered, the techniques may readily indicate to the surgeon whether vitreous is present or absent in the aspirated material and whether an occlusion has occurred.

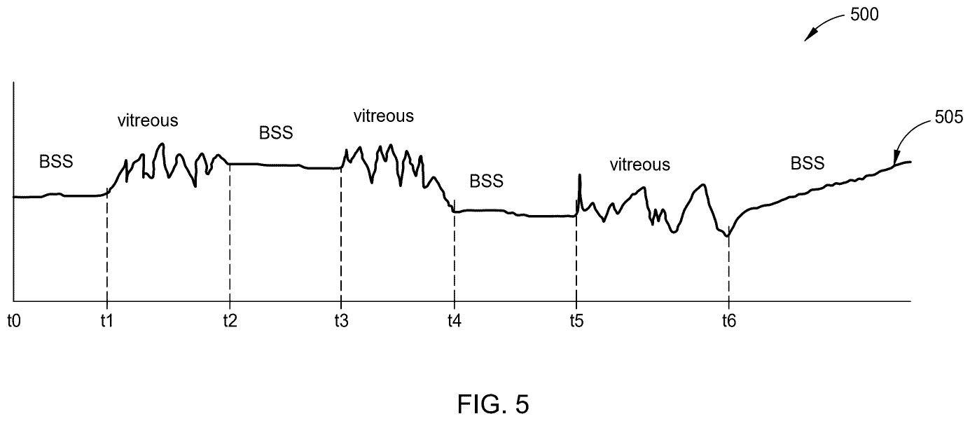

FIG. 5 illustrates an exemplary sequence of media detection during a surgical procedure, according to one or more embodiments. The features of FIG. 5 may be used in conjunction with other embodiments.

Diagram 500 includes a plot 505 representing a fluidic resistance along the aspiration path 360 over a period of time. During a surgical procedure, different media or compositions of media may be encountered (e.g., gases, thicker material) causing the response of the vacuum connections 350 to change. These changes may not match a static tubing model 320, resulting in a shift of the tubing model 320 and an inaccurate estimate of the fluidic resistance at the surgical instrument 110. When BSS (or other reference material) is aspirated, the fluidic resistance tends to have minimal variations. For example, between times t0 and t1, times t2 and t3, and times t4 and t5, the fluidic resistance is substantially constant (although the resistance levels for different time periods may differ from each other), and after time t6, the fluidic resistance exhibits a slow linear change.

When vitreous material is aspirated, the fluidic resistance tends to have greater variability, especially when the surgical instrument 110 is operated near the interface of vitreous and BSS (e.g., alternating between BSS and vitreous). For example, between times t1 and t2, times t3 and t4, and times t5 and t6, the fluidic resistance rapidly fluctuates (e.g., having higher rate(s) of change than when only BSS is encountered).

In some embodiments, the indication provided to the user may be based on a function of the fluidic resistance and the rate of change of the fluidic resistance. In one example, an increase in the fluidic resistance results in an indication that the probe is entering a region of more viscous material (e.g., vitreous), which may be independent of the amplitude of the fluidic resistance. In another example, the increase in the fluidic resistance may not result in an indication when the increase is less than a threshold rate. In another example, a highly variable fluidic resistance results in an indication that the probe is in a region of partial vitreous, which includes cases where the average fluidic resistance is relatively low.

The present disclosure may be embodied in other specific forms without departing from its spirit or essential characteristics. The described embodiments are to be considered in all respects only as illustrative and not restrictive. The scope of the present disclosure is, therefore, indicated by the appended Claims rather than by this Detailed Description. All changes which come within the meaning and range of equivalency of the Claims are to be embraced within their scope.

Reference throughout this specification to features, advantages, or similar language does not imply that all the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the present disclosure.

Reference throughout this specification to “one embodiment”, “an embodiment”, or similar language means that a particular feature, structure, or characteristic described in connection with the indicated embodiment is included in at least one embodiment of the present disclosure. Thus, the phrases “in one embodiment”, “in an embodiment”, and similar language throughout this specification may, but do not necessarily, all refer to the same embodiment.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein but are to be accorded the full scope consistent with the language of the claims.

What is claimed is:

1. A method of identifying a composition of an aspirated material during a surgical procedure, the method comprising:
controlling a surgical instrument to aspirate material through a probe of the surgical instrument, wherein the probe is fluidly connected with a vacuum source in an aspiration path;
estimating, during aspiration of the material, a flow rate of the material at the probe;
determining, using one or more sensors disposed along one or both of the aspiration path and an infusion path, a change in pressure across the probe during aspiration;
determining a fluidic resistance of the material using the change in pressure; and
providing feedback based, at least in part, on the fluidic resistance of the material and a reference value.

2. The method of claim 1, further comprising:
determining a fluidic viscosity of the material based on the fluidic resistance and a change in the flow rate,
wherein the feedback is based on a comparison of the fluidic viscosity of the material with the reference value.

3. The method of claim 2, wherein the reference value comprises a viscosity of a balanced salt solution.

4. The method of claim 1, wherein the probe comprises a vitrectomy probe, extrusion handpiece, or ultrasonic handpiece, and wherein the feedback indicates whether the material includes vitreous or lens material.

5. The method of claim 4, further comprising:
determining, based on the fluidic resistance of the material, an amount of vitreous or lens material included in the material,
wherein the feedback indicates the amount of vitreous included in the material.

6. The method of claim 1, wherein the aspiration path comprises tubing arranged between the surgical instrument and the vacuum source, the method further comprising:
accessing a model indicating compliance of the tubing; and
estimating, using the model, a flow rate component attributable to the compliance of the tubing,
wherein estimating the flow rate of the material at the probe comprises subtracting the flow rate component from an estimated pump flow rate.

7. The method of claim 6, further comprising:
receiving identification information for one or more components included in the aspiration path,
wherein accessing the model is based on the identification information.

8. The method of claim 1, further comprising:
providing, using an infusion source, a second material during aspiration of the material,
wherein estimating the flow rate of the material at the probe comprises determining an estimated flow rate as an inflow rate of the infusion source.

9. The method of claim 8, wherein estimating the flow rate of the material at the probe further comprises:
measuring an outflow rate at the vacuum source; and
comparing the estimated flow rate with the outflow rate.

10. The method of claim 8, wherein the aspiration path comprises tubing arranged between the probe and the vacuum source, the method further comprising:
accessing a model indicating compliance of the tubing; and
estimating, using the model, a flow rate component attributable to the compliance of the tubing,
wherein estimating the flow rate of the material at the probe further comprises subtracting the flow rate component from an initial value of the flow rate.

11. A surgical console comprising:
a vacuum source configured to fluidly connect, in an aspiration path, with a probe of a surgical instrument;
one or more processors; and
a memory storing instructions that, when executed by the one or more processors, enable performance of an operation comprising:
controlling the surgical instrument to aspirate material through the probe of the surgical instrument;
estimating, during aspiration of the material, a flow rate of the material at the probe;
determining, using one or more sensors disposed along one or both of the aspiration path and an infusion path, a change in pressure across the probe during aspiration;
determining a fluidic resistance of the material using the flow rate and the change in pressure; and
providing feedback based, at least in part, on the fluidic resistance of the material and a reference value.

12. The surgical console of claim 11, wherein the probe comprises a vitrectomy probe, extrusion handpiece, or ultrasonic handpiece, and wherein the feedback indicates whether the material includes vitreous or lens material.

13. The surgical console of claim 11, wherein the aspiration path comprises tubing arranged between the probe and the vacuum source, the operation further comprising:
accessing a model indicating compliance of the tubing; and
estimating, using the model, a flow rate component attributable to the compliance of the tubing,
wherein estimating the flow rate of the material at the probe comprises subtracting the flow rate component from an initial value of the flow rate.

14. The surgical console of claim 11, further comprising:
an infusion source configured to provide a second material during aspiration of the material,
wherein estimating the flow rate of the material at the probe comprises determining an estimated flow rate as an inflow rate of the infusion source.

15. The surgical console of claim 11, wherein:
the one or more processors are communicatively connected with one or both of a display and an input device configured to control operations of the surgical console based on received inputs, and
the feedback is provided using one or both of the display and the input device.
